(19) United States
(12) Patent Application Publication
(10) Pub. No.: US 2006/0212014 A1
(43) Pub. Date: Sep. 21, 2006

(54) Disposable absorbent article having removable portions

(76) Inventor: Bridget Ann Balogh, Menasha, WI (US)

Correspondence Address: KIMBERLY-CLARK WORLDWIDE, INC., 401 NORTH LAKE STREET, NEENAH, WI, 54956, US

(21) Appl. No.: 11/440,674

(22) Filed: May 24, 2006

Related U.S. Application Data
(63) Continuation of application No. 10/812,559, filed on Mar. 30, 2004.

(51) Int. Cl. A61F 13/15

(52) U.S. Cl. 604/385.11; 604/400; 604/385.01

(57) ABSTRACT

A disposable absorbent article is disclosed having a chassis with a front region, a back region and a crotch region. The crotch region connects the front region to the back region. The front and back regions are also secured together to form an absorbent article having a waist opening, a pair of leg openings and two side panels each located between the waist opening and one of the pair of leg openings. An absorbent is positioned in at least a portion of the crotch region. The absorbent article also has a removable portion formed in at least a portion of one of the side panels which intersects a portion of the circumference of one of the leg openings. The removable portion enables a portion of one of the side panels to be removed to customize the fit of the absorbent article to a user's anatomy.

### BACKGROUND OF THE INVENTION

Disposable absorbent articles are well known for their use in absorbing and retaining liquid and/or solid discharges from the human body. Infant diapers, child training pants and adult incontinence briefs, undergarments and refastenable underwear all fall into the category of disposable absorbent articles. In the case of adult incontinence garments, there are three primary styles of protective absorbent products that are commercially available today. The first style is an undergarment which is a substantially rectangular absorbent member which is positioned about the crotch region of a user and is then secured about the torso by a pair of elastic side straps. This style of undergarment provides essentially no side or hip protection. The second style is a brief which has a front region and a back region joined together by a crotch region. The brief also has side panels which fasten to the front region by a refastening mechanism to form a complete closure around the user's torso. The third style is a pull-on pant which appears similar to regular cloth underwear and has full side panels that extend from the waist opening down to each of the pair of leg openings. Users of such adult incontinence products, especially those who have moderate to heavy incontinence, must choose between multiple products. This forces some users to buy some combination of the above-identified styles of absorbent undergarments to meet their everyday needs. Sometimes, a pull-on pant style absorbent undergarment with full side/hip coverage is needed to assure the user that no fluid leakage will occur. This is especially true for undergarments that are worn when the user is active playing sports or during the night as the user is sleeping. It has also been found that in some countries and in certain geographical areas, the temperature and climate are such that a full side/hip coverage can be uncomfortable. In these situations, an absorbent undergarment having only a partial side/hip coverage may be more appropriate. Furthermore, the circumferences of some user's right and left thighs vary and this can cause the leg opening of the undergarment to be tighter or looser around one of the user's thighs. Likewise, some users wear an artificial limb or prosthesis and it would be beneficial if the user could adjust the leg opening to obtain a more comfortable fit.

Now a disposable absorbent article has been invented that has at least one removable portion that can be removed to customize the fit of the absorbent article to a user's anatomy.

### SUMMARY OF THE INVENTION

Briefly, this invention relates to a disposable absorbent article having a chassis with a front region, a back region and a crotch region. The crotch region connects the front region to the back region. The front and back regions are also secured together to form an absorbent article having a waist opening, a pair of leg openings and two side panels each located between the waist opening and one of the pair of leg openings. An absorbent is positioned in at least a portion of the crotch region. The absorbent article also has a removable portion formed in at least a portion of one of the side panels which intersects a portion of the circumference of each of the leg openings. The removable portion enables a portion of one of the side panels to be removed to customize the fit of the absorbent article to a user's anatomy.

### BRIEF DESCRIPTION OF THE DRAWINGS

FIG. 1 is a perspective view of a disposable absorbent article having a plurality of removable portions formed in the front region of the article.

FIG. 2 is a cross-sectional view of FIG. 1 taken along line 2-2 showing an exterior layer, the absorbent and an interior layer secured together.

FIG. 3 is a side view of the disposable absorbent article shown in FIG. 1 depicting the plurality of removable portions present in the front region of the article.

FIG. 4 is a side view of an alternative embodiment of a disposable absorbent article showing a plurality of removable portions present in only the back region of the article.

FIG. 5 is a side view of still another alternative embodiment of a disposable absorbent article showing a plurality of removable portions present in both the front and back regions of the article.

FIG. 6 is a partial view of a disposable absorbent article depicting a plurality of removable portions present in the front region of the article and leg elastics extending from the side seam to a location where the removable portions intersect the leg opening.

FIG. 7 is a cross-sectional view of FIG. 6 taken along line 7-7 showing three strands of leg elastics sandwiched between an exterior layer and an interior layer.

FIG. 8 is an enlarged view of the circled area on FIG. 6 showing a portion of the side seam with a tear line extending up to the side seam but not extending into the ultrasonic bond pattern.

FIG. 9 is an alternative embodiment of the enlarged view of the circled area on FIG. 6 showing a portion of the side seam with a tear line extending into the side seam and across the ultrasonic bond pattern.

FIG. 10 is still another embodiment of the enlarged view of the circled area on FIG. 6 showing a portion of the side seam with a tear line extending into the side seam but not present in the ultrasonic bond pattern.

FIG. 11 is still another embodiment of the enlarged view of the circled area on FIG. 6 showing a portion of the side seam having an oversized bond area with a tear line intersecting the oversized bond area.

FIG. 12 is a perspective view of a disposable absorbent article showing a plurality of removable portions having a semi-circular configuration formed in the front region of the article and elastic surrounding essentially the entire circumference of each of the leg openings.

### DETAILED DESCRIPTION

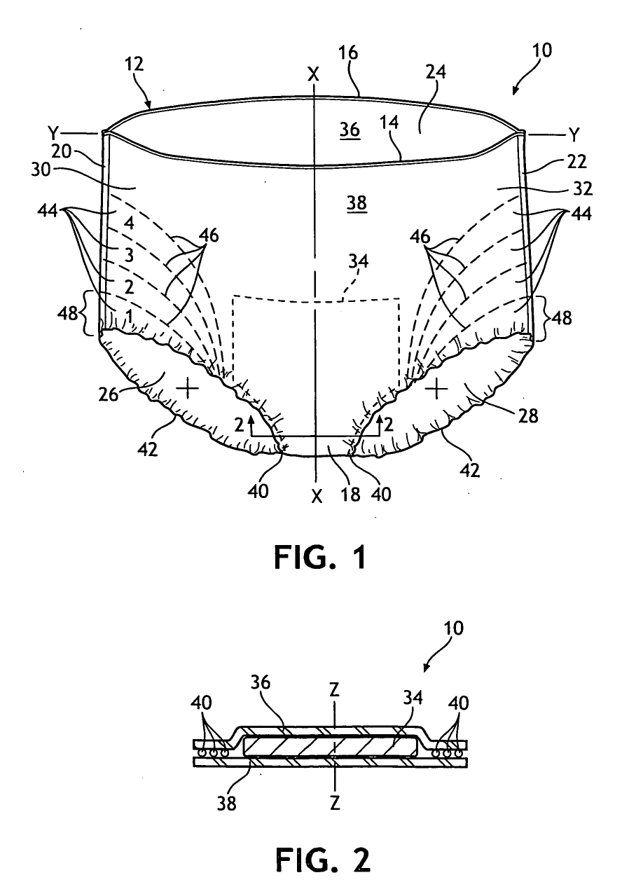

Referring to FIG. 1, a disposable absorbent article 10 for absorbing human discharge is shown. A “disposable absorbent article” as used herein is an absorbent article that is intended to be worn by humans, including infants, toddlers or adults, which is designed for single use or temporary use and is meant to be disposed of after being used once instead of being laundered or dry cleaned for re-use. The disposable absorbent article 10 is designed to absorb and/or retain one or more bodily discharges of waste material such as urine, perspiration, excrement, feces, menses, menstrual fluid, as well as other liquid and/or solid waste. The disposable absorbent article 10 is shown as a tubular shaped undergarment designed to be worn around the torso of human body. The disposable absorbent article 10 can be a diaper worn by infants, a training pant worn by toddlers or an incontinence garment normally worn by teenagers or adults. The disposable absorbent article 10 can be a pull-on style pant undergarment which is similar to regular cloth underwear or a diaper or brief style undergarment that is positioned around a user's torso and then is secured in place by one or more fasteners. The fasteners can include refastenable tapes, hook and loop engaging fasteners, VELCRO® type fasteners, buttons and button holes, safety pins, etc. VELCRO® is a registered trademark of Velcro USA, Inc., having an office at 406 Brown Avenue, Manchester, N.H. 03103.

Referring to FIGS. 1-3, the disposable absorbent article 10 has a chassis 12 that includes a front region 14, a back region 16 and a crotch region 18. The disposable absorbent article 10 has a longitudinal axis X-X and a transverse axis Y-Y, see FIG. 1, and a vertical axis Z-Z, see FIG. 2. The crotch region 18 connects the front region 14 to the back region 16. The front and back regions, 14 and 16 respectively, are secured together by a pair of side seams 20 and 22 to form an absorbent article having a waist opening 24 and a pair of leg openings 26 and 28. The waist opening 24 is sized to fit around the waist of an average user of the disposable absorbent article 10. A waist band, not shown, can be constructed around the waist opening 24 to ensure that the disposable absorbent article 10 remains firmly in place around a user's torso. The side seams 20 and 22 extend from the waist opening 24 to one of the leg openings 26 or 28. Each of the side seams 20 and 22 can be formed by a sonic bond, by an ultrasonic bond, by an adhesive or by other means of attachment known to those skilled in the art. Each of the pair of leg openings 26 and 28 is sized to fit around the upper thighs of a user. The disposable absorbent article 10 also has two side panels 30 and 32 located between the waist opening 24 and one of the pair of leg openings, 26 and 28 respectively. Each of the side panels 30 and 32 also include one of the side seams 20 and 22.

The disposable absorbent article 10 also has an absorbent 34 positioned in at least a portion of the crotch region 18. The absorbent 34 can cover the entire crotch region 18 if desired and can also extend into either or both of the front and back regions, 14 and 16 respectively. The amount of absorbent 34 present in any of the three regions of the disposable absorbent article 10 can vary depending upon the use of the absorbent article 10 and the size and age of the user who will wear the disposable absorbent article 10. For example, a diaper for use by an infant may not require the absorbent capacity needed by an adult incontinence undergarment.

The absorbent 34 can be formed from natural or synthetic materials. The absorbent 34 can be made from cellulosic fibers, wood pulp, textile fibers or other absorbent materials known to those skilled in the art. Superabsorbents, in solid form and in the shape of small particles, granules, flakes, etc., can be mixed in with the absorbent material to increase the absorbent capacity of the absorbent 34.

In FIG. 2, the absorbent 34 is shown secured or sandwiched between a liquid pervious bodyside liner 36 and a liquid-impervious outer cover 38. The absorbent 34 is shown sealed within the liquid pervious bodyside liner 36 and the liquid-impervious outer cover 38. The liquid pervious bodyside liner 36 is located nearest to the human body, adjacent to the skin of the user, and can be formed from a woven or non-woven material that will readily allow liquid or fluids to pass therethrough. The bodyside liner 36 is normally a very thin web that can be formed from natural or synthetic fibers, with or without apertures formed therein. A spunbond and a bonded carded web are two materials that work well as a bodyside liner 36. “Spunbond” is manufactured and sold commercially by Kimberly-Clark Corporation, having an office at 401 North Lake Street, Neenah, Wis. 54956.

The liquid-impervious outer cover 38 is located on the exterior of the disposable absorbent article 10, away from the skin of the user. The liquid-impervious outer cover 38 is formed from a material which will restrict fluid from penetrating or passing therethrough so as to prevent the outer clothing of the wearer from becoming soiled. Desirably, the outer cover 38 has a soft feel so as not to chafe the inner thighs of the wearer. The outer cover 38 can also be formed from natural or synthetic fibers. The outer cover 38 can be formed from a material that is not noisy when squeezed or wrinkled so that the disposable absorbent article 10 remains discreet. The outer cover 38 can also be formed from a breathable material. The outer cover 38 can further be formed from a laminate where one layer of the laminate is liquid-impervious. Examples of various materials that can be used as the outer cover 38 include a polyolefin, such as polypropylene or polyethylene; a liquid-impervious layer bonded to a spunbond; and a thermoplastic material bonded to a spunbond. Other materials known to those skilled in the art can also be utilized.

It should also be noted that the absorbent 34 can also be formed as an absorbent assembly, not shown, that includes an absorbent secured between two exterior layers. Such an absorbent assembly can then be secured to the inside of the crotch region 18 of the disposable absorbent article 10. When such an absorbent assembly is utilized, the disposable absorbent article 10 can be constructed such that the front, back and crotch regions, 14, 16 and 18 respectively, can be formed from both the liquid pervious bodyside liner 36 and the liquid-impervious outer cover 38. Alternatively, one or more of the front, back and/or crotch regions, 14, 16 and 18 respectively, could be constructed of only one of the layers 36 or 38, if desired.

Still referring to FIG. 2, one can see elastic strands 40 positioned laterally outward of the absorbent 34 and secured between the liquid pervious bodyside liner 36 and the liquid-impervious outer cover 38. Three elastic strands 40 are depicted on each side of the absorbent 34 although a fewer or a greater number of elastic strands 40 could be used. The elastic strands 40 can be formed from LYCRA®. LYCRA® is a registered trademark of E. I. Du Pont De Nemours & Co., having an office at 1007 Market Street, Wilmington, Del. 19898. The diameter and/or cross-sectional configuration of the elastic strands 40, the decitex (weight in grams per 10,000 meters of a strand) of the elastic strands 40, and tension imparted into the elastic strands 40 can all be varied to suit one's particular product needs. The elastic strands 40 form the crotch elastics on the disposable absorbent article 10 and can cooperate with leg elastics 42 formed about a portion of the leg openings 26 and 28, see FIG. 1.

Returning again to FIG. 1, the leg elastics 42 are shown cooperating with the crotch elastics 40 to encircle essentially the entire circumference of each of the leg openings 26 and 28. Alternatively, the leg elastics 42 can be applied such that they cooperate with the crotch elastics 40 to surround a portion of the circumference of the leg openings 26 and 28. Usually the leg elastics 42 and the crotch elastics 40 cooperate to cover from between about 50% to 100% of the circumference of each of the leg openings 26 and 28. More desirably, the leg elastics 42 and the crotch elastics 40 cooperate to cover from between about 75% to 100% of the circumference of each of the leg openings 26 and 28. Alternatively, it is possible to apply the leg elastics 42 such that small gaps exist between them and the crotch elastics 40.

The disposable absorbent article 10 further includes one or more removable portions 44 each positioned adjacent to one of the pair of leg openings 26 and 28. The removable portions 44 should be void of the absorbent 34. Another way of stating this is to say that the removable portions 44 should not intersect or overlap the absorbent 34. One reason for this is that the absorbent 34 is an expensive component and is needed to prevent fluid leakage. One would not want to decrease or limit the amount of absorbent 34 remaining in the disposable absorbent article 10 when the removable portions 44 are detached.

In FIG. 1, two sets of four removable portions 44 are shown being formed in the front region 14. Each set is aligned on opposite sides of the longitudinal axis X-X. Although two sets of four removable portions 44 are shown, one could construct a disposable absorbent article 10 with a set of one, two, three, four or more removable portions 44. As many as twenty removable portions 44 could be employed in a large size incontinence undergarment, if desired. It is also possible to construct a disposable absorbent article 10 with only one removable portion 44 aligned adjacent to only one of the leg openings 26 or 28. Although this asymmetric design might appear strange, there may be reasons why such a design would be commercially feasible.

In FIG. 1, the four removable portions 44 in each set are labeled 1, 2, 3 and 4 for discussion purposes only, with number 1 being aligned adjacent to one of the leg openings 26 or 28. As the number increases, that respective removable portion 44 is positioned farther away from one of the leg openings 26 or 28 and closer to the waist opening 24. Each of the removable portions 44 is formed in at least a portion of one of the side panels 30 and 32. Each removable portion 44 is part of the front region 14 and is constructed of the same material that is used to construct the front region 14. The removable portions 44 are established by forming a tear line 46 in the disposable absorbent article 10. The tear line 46 can be formed as a perforation line, a plurality of separation points, a score line, a line of weakness, zones of weakness, a breakaway line or areas, a chain stitch, etc. A “chain stitch” is a stitch formed in the material such that when an end of the stitching is pulled, the stitch unravels and the material separates. Each tear line 46 can pass partially or completely through the thickness of both the liquid permeable bodyside liner 36 and the liquid-impermeable outer cover 38. Each tear line 46 can be linear or non-linear in shape or configuration. Non-linear shapes can include curved or arcuate profiles, a saw tooth profile, a semi-circular profile, a zigzag profile, a sinusoidal profile, or any other geometrical profile that is not a straight line. Desirably, each tear line 46 will have an arcuate or semi-circular configuration.

Still referring to FIG. 1, each tear line 46 establishes a removable portion 44 having a rounded trapezoidal or partial crescent shape. By “crescent shape”, it is meant an appearance similar to the figure of the moon as it appears in its first or last quarter with concave and convex edges terminating at two spaced apart points. By a “partial crescent shape”, it is meant a portion of a full crescent shape. Each of the removable portions 44 has a tear line 46 that start or intersect at a point located on the circumference of one of the leg openings 26 or 28 and extends in a continuous manner toward one of the side seams 20 or 22. All of the removable portions 44 can start at approximately the same point on the circumference of one of the leg openings, 26 and 28 respectively, if desired. In FIG. 1, the tear lines 46 are shown extending upward and outward, away from the longitudinal axis X-X, and toward one of the side seams 20 and 22. The tear lines 46 can terminate adjacent to, at or in the respective side seam 20 or 22. A more detailed description of this feature will be explained shortly. The removable portions 44 function to enable the user to remove and discard one or more removable portions 44 to customize the fit of the disposable absorbent article 10 to the user's anatomy. For example, an adult suffering from incontinence may have a right thigh that is slightly larger in circumference than the left thigh. In this case, the user could detach removable portion 1 located on the right side of the disposable absorbent article 10. This action will cause the leg opening 26 to be made larger and make the disposable absorbent article 10 more comfortable to wear.

It should be noted that one or more of the removable portions 44 can be disconnected from the disposable absorbent article 10 either sequentially or simultaneously. Likewise, it is not necessary that each side of the disposable absorbent article 10 match or be symmetric once a number of the removable portions 44 have been disconnected. For example, one removable portion 44 can be detached from the right side of the disposable absorbent article 10 and two removable portions 44 could be detached from the left side of the same article 10.

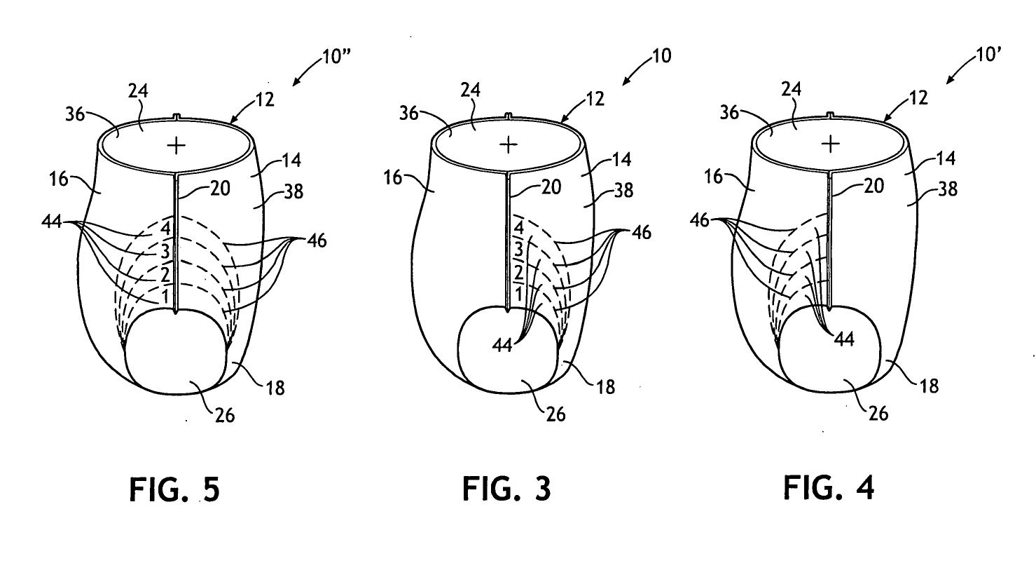

Referring to FIGS. 1 and 3, one will notice that each of the tear lines 46 extend from one of the respective leg openings 26 or 28 to one of the side seams 20 or 22. The tear lines 46 could be constructed to pass or extend through the elastic strands forming the crotch elastics 40 or the leg elastics 42, if desired. Each of the tear lines 46 for the removable portions 44 are numbered 1, 2, 3 and 4 and each has a different length. As one moves away from the leg openings 26 and 28 and toward the waist opening 24, the tear lines 46 become longer in length. For example, the tear line 46 separating removable portion 1 from removable portion 2 is aligned closest to the leg opening 26 or 28 and can be made to,have a predetermined length. Each tear line 46 spaced farther away from this first tear line 46 and closer to the waist opening 24 will have a longer length. Likewise, the tear line 46 separating removable portion 2 from removable portion 3 is shorter in length than the tear line 46 which separates removable portion 3 from removable portion 4. The exact length of each tear line 46 will be determined by a number of factors, including the overall size of the disposable absorbent article 10, the profile of the various tear lines 46, the beginning and ending points of each of the tear lines 46, and the rise of the tear lines 46 away from one of the leg openings 26 or 28 and toward the waist opening 24. Other factors that can determine the length of each of the tear lines 46 can be the size and surface area of the removable portion 44.

Just as the tear lines 46 have different lengths, the surface area of each of the removable portions 44 can also be different. Although the surface area of one or more of the removable portions 44 could be sized to be the same, most likely they will be different. In this regard, the removable portion 44 located closest to the leg openings 26 or 28 can have the smallest surface area and each subsequent removable portion 44, located farther away from the leg openings 26 or 28 and closer to the waist opening 24, can have a larger surface area. For example, the surface area of removable portion 1 can be smaller than the surface area of removable portion 2. Likewise, the surface area of removable portion 2 can be smaller than the surface area of removable portion 3. The actual surface area of each removable portion 44 can be varied to suit one's particular disposable absorbent article 10.

Once the tear line 46 is torn, a lower portion of the side seam 20 or 22 is then broken. The lower portion of removable portion 1 is denoted as 48 in FIG. 1. The tear line 46 can extend into or across the width of the side seam 20 or 22 so as to enable the lower portion of the side seam 20 or 22 to easily tear. Once the removable portion 44 has been separated from the disposable absorbent article 10, it should be properly disposed of in a trash or waste container. By removing one or more of the removable portions 44 from the disposable absorbent article 10, one can enlarge the leg openings 26 and 28 and also reduce the full side or hip coverage of the user's torso when such is not warranted. For example, on a very hot or muggy day, the user may feel more comfortable when a greater percentage of the side panels 30 and 32 are removed.

Turning now to FIG. 4, an alternative embodiment of a disposable absorbent article 10′ is shown. In this embodiment, a plurality of removable portions 44 is present in the back region 16 instead of in the front region 14. Each of the tear lines 46 will start or intersect at a point located on the circumference of one of the leg openings 26 or 28 and extend in a continuous manner toward one of the side seams 20 or 22. In FIG. 4, the tear lines 46 are shown extending upward and inward, toward the longitudinal axis X-X, and toward one of the side seams 20 and 22. The tear lines 46 can terminate adjacent to, at or in the respective side seam 20 or 22. The tear lines 46 can also be formed in the side seams 20 and 22 such that they extend down to or towards the respective leg opening 26 or 28.

Turning now to FIG. 5, a third embodiment of a disposable absorbent article 10″ is shown. In this embodiment, a plurality of removable portions 44 is present in the front and the back regions, 14 and 16 respectively, of the disposable absorbent article 10″. Each of the tear lines 46 will start or intersect at a point located on the circumference of one of the leg openings 26 or 28 and extend across one of the side seams 20 or 22 and terminate at a spaced apart location on the same leg opening 26 or 28. In this embodiment, each of the removable portions 44 is approximately twice as large as those shown in FIGS. 3 or 4. However, the function of removing one or more of the removable portions 44 from the disposable absorbent article 10″ is the same as stated above with reference to FIGS. 1, 3 and 4. By removing one or more of the removable portions 44, a customized fit of the disposable absorbent article 10″ to the user's anatomy is obtained. Furthermore, as stated above with reference to FIG. 4, the tear lines 46 can also be formed in the side seams 20 and 22 such that they extend down to or towards the respective leg opening 26 or 28. The tear lines 46 can also be formed in the side seams 20 and 22 such that they extend down to or towards the respective leg opening 26 or 28.

Turning now to FIG. 6, a portion of a disposable absorbent article 10″′ is shown which is similar to the design of the disposable absorbent article 10 depicted in FIG. 1 except for one difference. In FIG. 6, it should be noted that the remaining half of the disposable absorbent article 10″′, not shown, is similar in appearance to the portion that is shown. For purposes of discussion, only those numerals listed on the portion of the disposable absorbent article 10″′ that are shown will be described. In FIG. 6, the four removable portions 44, labeled 1, 2, 3 and 4, are all located in the front region 14 but the crotch elastics 40 and the leg elastics 42 do not extend around essentially the entire circumference of each of the leg openings 26 and 28. Instead, the elastic strands forming the leg elastic 42 extend from one of the side seams 20 or 22 inward toward the longitudinal axis X-X but they stop short of the crotch elastics 40. A gap 50 is shown separating the leg elastic 42 from the crotch elastic 40. The gap 50 can be small or large in dimension. The gap 50 can vary in dimension from about 0.1 inches (about 0.25 cm) to about 3 inches (about 7.5 cm) when measured with the disposable absorbent article 10″′ being opened and stretched out flat on a horizontal surface. Desirably, the gap 50 can vary in dimension from about 0.1 inches (about 0.25 cm) to about 2 inches (about 5 cm) when measured with the disposable absorbent article 10″′ being opened and stretched out flat on a horizontal surface. More desirably, the gap 50 can vary in dimension from about 0.2 inches (about 0.5 cm) to about 1 inch (about 2.54 cm) when measured with the disposable absorbent article 10″′ being opened and stretched out flat on a horizontal surface.

In FIG. 6, one can see that no leg elastics 42 are present in the removable portions 44 along the circumference of the leg opening 26. This is by design. In FIG. 6, the tear lines 46 are also spaced away from the crotch elastics 40 and the leg elastics 42. By doing so, it may make it easier for the user to initiate breaking the tear lines 46. The absence of leg elastics 42 in the front region 14 will not materially affect the function of the disposable absorbent article 10″′ if the front, back and/or crotch regions, 14, 16 and 18 respectively, are formed from an elastomeric material. By an “elastomeric material” it is meant a material that is capable of being stretched in at least one direction and has the ability to contract or return back towards its original length. The contraction does not have to return the material back 100% to its original length. The elastomeric material can be a woven or nonwoven material. The front, back and/or crotch regions, 14, 16 and 18 respectively, can be formed from a breathable or a non-breathable elastomeric material. An elastomeric polyolefin, such as polypropylene or polyethylene can be used. Other useful materials include an elastomeric spunbond and an elastomeric bonded carded web. An elastomeric, metallocene polypropylene works very well since it has a soft feel and can be easily ultrasonically bonded to itself.

It should be noted that one can align and secure one or more elastic strands (not shown) along each of the tear lines 46 so that as a removable portion 44 is removed, elastic strands will still be present around the entire leg opening 26.

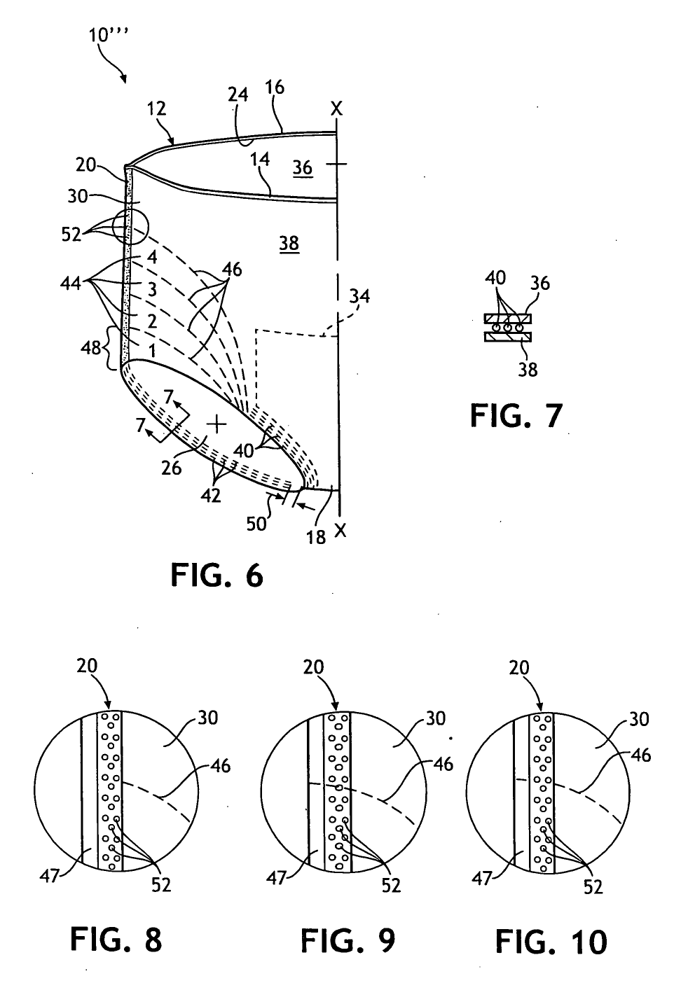

Referring now to FIG. 7, a cross-sectional view of the three elastic strands forming the leg elastics 42 is shown. In FIG. 7, the three elastic strands 42 are secured between the liquid permeable bodyside liner 36 and the liquid-impermeable outer cover 38. The elastic strands 42 can be attached using a hot or cold melt adhesive. Other means of securing the leg elastics 42 between the two outer layers, 36 and 38 respectively, are known to those skilled in the art and can also be utilized.

Referring now to FIGS. 8-11, four views of a portion of a side seam are shown which clearly depict four various patterns that can be used. In FIG. 8, a first configuration is shown wherein the tear line 46 is formed in the side panel 30 and extends up to the inside edge of the side seam 20. However, the tear line 46 does not extend into the side seam 20. Accordingly, the tear line 46 does not contact or cross the bonds 52 which seals the front region 14 to the back region 16, see FIG. 6. One will also notice a small strip of material 47 is present to the left of the bonds 52 and forms a fringe 47. The fringe 47 does not have to be present but when it is, the outer edge of the side seams, 20 and 22 respectively, tends to be softer to the touch.

In FIG. 9, a second configuration is shown wherein the tear line 46 is formed in the side panel 30 and extends across the width of the side seam 20. The tear line 46 does contact and crosses over the bonds 52. The tear line 46 also extends into the fringe 47.

In FIG. 10, a third configuration is shown wherein the tear line 46 is formed in the side panel 30 and extends across a portion of the width of the side seam 20. The tear line 46 does not cross the bonds 52 but is present in the fringe 47.

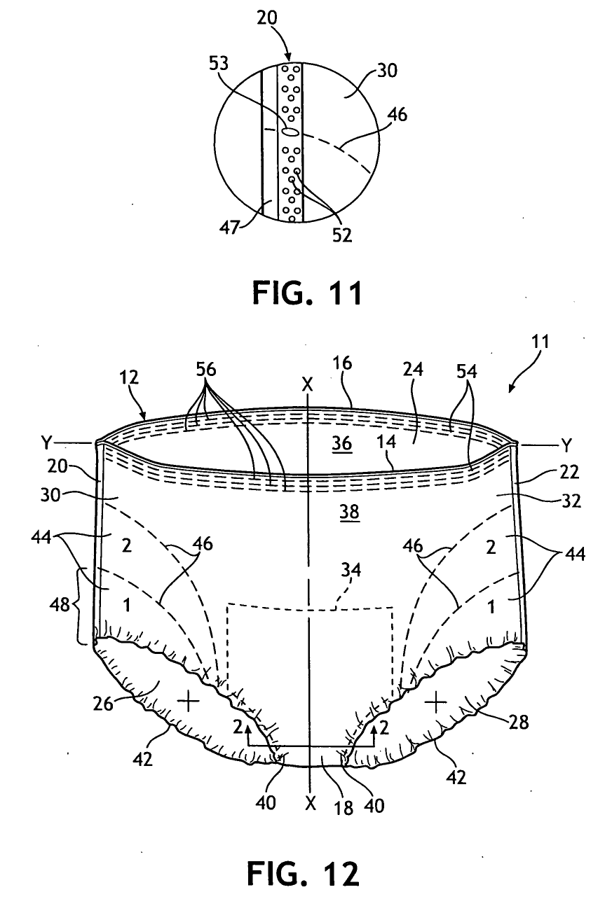

In FIG. 11, a fourth configuration is shown wherein the bonds 52 include at least one oversized bond 53. The tear line 46 is formed in the side panel 30 and extends across a portion of the width of the side seam 20 and extends into the fringe 47. The tear line 46 intersects the location of the oversized bond 53. The tear line 46 does not actually contact the perimeter of the oversized bond 53 but is aligned adjacent to its perimeter. Any of these four configurations, as well as others known to those skilled in the art can be used, to ensure that the removable portions 44 will break away and be torn free from the side seams 20 and 22. It should be noted that the strength of the side seams 20 and 22, the material from which the side seams 20 and 22 are constructed, the width of the side seams 20 and 22, as well as the pattern and actual size and shape of the tear lines 46 can all influence the amount of force needed to break and detach the removable portions 44 from the disposable absorbent article 10, 10′, 10″ and 10″′.

Lastly, referring to FIG. 12, another embodiment of a disposable absorbent article 11 is shown which is similar to that depicted in FIG. 1, except for a couple of differences. In FIG. 12, one will notice that only two removable portions 44, labeled 1 and 2, are present in each of the side panels 30 and 32. A second difference is that each of the tear lines 46 has a semi-circular profile or configuration. The reduced number of removable portions 44 together with the semi-circular configuration of each tear line 46 means that the surface area of removable portion 1 can be less than, equal to or greater than the surface area of removable portion 2. However, as described with reference to FIG. 1, the length of the tear line 46 aligned closest to the leg openings 26 and 28 will normally be shorter in length than a tear line 46 spaced farther away from the leg openings 26 and 28, and closer to the waist opening 24. A third difference in FIG. 12 is that a waist band 54 is formed in the front and back regions 14 and 16 of the disposable absorbent article 11. The waist band 54 contains from 1 to about 12 elastic strands 56 secured between the liquid permeable bodyside liner 36 and the liquid-impermeable outer cover 38. The waist band 54 is aligned essentially parallel to the waist opening 24 and has a width, measured parallel to the longitudinal axis X-X, of from between 0.25 inches (about 0.6 cm) to about 2 inches (about 5 cm). Desirably, the waist band 54 has a width of about 1 inch (about 2.54 cm).

While the invention has been described in conjunction with several specific embodiments, it is to be understood that many alternatives, modifications and variations will be apparent to those skilled in the art in light of the aforegoing description. Accordingly, this invention is intended to embrace all such alternatives, modifications and variations that fall within the spirit and scope of the appended claims.

1. A disposable absorbent article comprising:
a) a chassis having a front region, a back region and a crotch region, said crotch region connecting said front region to said back region, said front and back regions being secured together to form an absorbent article having a waist opening, a pair of leg openings each having a circumference, and two side panels each located between said waist opening and one of said pair of leg openings;
b) an absorbent positioned in at least a portion of said crotch region; and
c) a removable portion formed in at least a portion of one of said side panels which intersects a portion of the circumference of one of said leg openings and enables a portion of one of said side panels to be removed to customize the fit of said absorbent article to a user's anatomy, and said removable portion extending from said front region adjacent to one of said pair of leg openings to said back region and terminates adjacent to said same leg opening.

2. The disposable absorbent article of claim 1 wherein said removable portion is joined to one of said side panels by a tear line.

3. The disposable absorbent article of claim 2 wherein said tear line has an arcuate configuration.

4. The disposable absorbent article of claim 2 wherein said tear line is a perforation line.

5. The disposable absorbent article of claim 1 wherein said removable portion extends from said front region adjacent to one of said pair of leg openings to said back region and terminates adjacent to said same leg opening, and there are at least two removable portions present in each of said side panels.

6. The disposable absorbent article of claim 1 wherein there is at least one removable portion present in each of said side panels and each of said removable portions is joined to one of said side panels by a tear line.

7. The disposable absorbent article of claim 1 wherein there are at least two removable portions present in each of said side panels.

8. The disposable absorbent article of claim 7 wherein each of said removable portions in each of said side panels has a different surface area.

9. The disposable absorbent article of claim 7 wherein each of said removable portions is joined to one of said side panels by a tear line and said tear line aligned closest to one of said leg openings in each of said side panels has a predetermined length and said tear line aligned farther away from said leg opening has a longer length.

10. A disposable absorbent article comprising:
a) a chassis having a front region, a back region and a crotch region, said crotch region connecting said front region to said back region, said front and back regions being secured together by a pair of side seams to form an absorbent article having a waist opening, a pair of leg openings each having a circumference, and two side panels each located between said waist opening and one of said pair of leg openings;
b) an absorbent positioned in at least a portion of said crotch region; and
c) at least one removable portion formed in at least a portion of each of said side panels, each removable portion extending from a point on the circumference of one of said pair of leg openings to said respective side seam, and each removable portion enabling a portion of each of said side panels to be removed to customize the fit of said absorbent article to a user's anatomy.

11. The disposable absorbent article of claim 10 wherein each of said removable portions is joined to one of said side panels by a tear line.

12. The disposable absorbent article of claim 11 wherein elastic strands surround at least a portion of each of said pair of leg openings and said removable portion positioned adjacent to each of said leg openings has a tear line that extends through said elastic strands.

13. The disposable absorbent article of claim 10 wherein at least two removable portions are formed in at least a portion of each of said side panels and said removable portion located closest to one of said leg openings in each of said side panels has a smaller surface area than said removable portion located farther away from one of said leg openings.

14. The disposable absorbent article of claim 10 wherein said removable portions are void of said absorbent.

15. The disposable absorbent article of claim 10 wherein there are at least three removable portions present in each of said side panels.

16. A disposable absorbent article comprising:
a) a chassis having a front region, a back region and a crotch region, said crotch region connecting said front region to said back region, said front and back regions being secured together by a pair of side seams to form an absorbent article having a waist opening, a pair of leg openings each having a circumference, and two side panels each located between said waist opening and one of said pair of leg openings;
b) an absorbent positioned in at least a portion of said crotch region; and
c) at least three removable portions formed in at least a portion of each of said side panels, each removable portion extending from a point on the circumference of one of said pair of leg openings across one of said side seams and terminating at a spaced apart location on said same leg opening, and each removable portion enabling a portion of each of said side panels to be removed to customize the fit of said absorbent article to a user's anatomy.

17. The disposable absorbent article of claim 16 wherein each of said removable portions is joined to one of said side panels by a tear line.

18. The disposable absorbent article of claim 17 wherein each of said tear lines has a semi-circular configuration.

19. The disposable absorbent article of claim 16 wherein each of said removable portions formed in each of said side panels has a different surface area.

20. The disposable absorbent article of claim 16 wherein said removable portion located closest to one of said leg openings has a smaller surface area than each of said other removable portions located farther away from said leg opening.
